(19) United States
(12) Patent Application Publication
(10) Pub. No.: US 2008/0050274 A1
(43) Pub. Date: Feb. 28, 2008

(54) Process for treating medical waste

(76) Inventor: Brian Ozier, Athens, AL (US)

Correspondence Address: Donald C. Simpson, 1216 Borton Landing Road, Moorestown, NJ, 08057-3055, US

(21) Appl. No.: 11/810,803

(22) Filed: Jun. 7, 2007

Related U.S. Application Data
(60) Provisional application No. 60/811,724, filed on Jun. 7, 2006.

(51) Int. Cl. A61L 2/07

(52) U.S. Cl. 422/26

(57) ABSTRACT

An improved, but less costly method for disposing of infectious personal waste, as well as pathological waste and equipment which can be configured for large municipal type use employing continuous process techniques, or for smaller, batch type, stand-alone processors for individual sites such as hospitals.

The benefit of the filing date of Provisional Patent Application 60/811,724 filed Jun. 7, 2006 is claimed herein.

### FEDERALLY SPONSORED RESEARCH

This invention was not the subject of any federally sponsored research or development.

### BACKGROUND OF THE INVENTION

The disclosure of U.S. Pat. No. 5,217,688 (“Process for the Disposal of Medical Waste”), including the three figures, is incorporated herein by reference. The aforementioned patent describes both a process and an apparatus useful for reducing medical waste to a sterile “ash” in a carefully controlled autoclave having disintegration equipment contained therein. The process comprises the steps of:

A. providing a pressure vessel containing rotatable disintegration means, a closure means for said pressure vessel, and connected to said pressure vessel an entrained solids separator, a liquid condenser, a condensate receiver, a vacuum pump, and a filter of an activated carbon type;

B. introducing medical waste to be treated into said pressure vessel containing rotatable disintegration means;

C. sealing said pressure vessel with said closure means against the uncontrolled release of contents;

D. locking said closure means in a manner to preclude accidental or casual opening of said pressure vessel prior to the sterilization of the contents;

E. activating said rotatable disintegration means to effect a substantial disintegration of the contents of said pressure vessel;

F. heating the contents of said pressure vessel to a temperature in the range of about 245 to 270 degrees Fahrenheit and maintaining the contents at a temperature in said range for a period sufficient to destroy any micro-organism content of said medical waste;

G. terminating the heating of the contents of said pressure vessel;

H. drawing a vacuum on said pressure vessel through said entrained solids separator, said liquid condensor and said condensate receiver by means of said vacuum pump and passing the exhaust stream from said vacuum pump through said filter, and

I. discharging the contents of said pressure vessel; and further providing that all of the steps of said process other than the initial loading of the pressure vessel with medical waste and the discharge of medical waste therefrom are controlled by automatic control means provided with recorder means to provide a verifiable record of the sterilization of the contents of said pressure vessel.

In the preferred embodiment, heating step F comprises steam injection, and a vacuum of at least 25 inches of water is drawn on the pressure vessel following locking step D and prior to heating step F.

In all such processes, any steps which can reduce the time and energy requirements will provide a considerable financial savings as well, and encourage the use of such processes and equipment for the disposal of medical waste. Accordingly, the medical waste processing described in Parent No. 5,217,688 has been optimized. A vacuum is pulled in the closed vessel for one minute at the beginning of the cycle to remove as much air as possible. Then the vacuum is shut off and live steam is injected into the bottom of the vessel to heat up the contents to about 272° F. Knife action is then started at a speed of 1,750 r.p.m. and subsequently increased to 3,500 r.p.m.

### DESCRIPTION OF THE PRESENT INVENTION

The mode of operation of the aforementioned invention is now modified, although still using the same apparatus, to create a flushing action by having the steam injection into the bottom of the vessel(Step F), the application of vacuum from the top of the vessel (Step H), and the knife action (Step E) all occur simultaneously. When steam is visible in the exhaust line, typically after about 40 seconds, the vacuum is shut off, while the steam injection is continued for typically another 6 minutes, plus or minus 30 seconds, to achieve the required sterility of vessel before it is shut off. During this heat-up time, the knives keep running at about 1200 to about 2400 r.p.m., and preferably about 1,750 r.p.m. The typical vacuum for this improved process is at least 25 inches of water. With this system, the contents of the treatment vessel should be at least 250 degrees F. but below 290 degrees F. The lower temperature must be at least sufficient to destroy the microbiological content of the vessel and the upper temperature should be below the temperature at which plastics and other similar components of the vessel contents will decompose. Originally it was believed that the upper temperature should not exceed about 270 degrees F. but it has now been determined that the length of time at the higher temperature is a significant factor in the plastic decomposition process. Since the improved process of the present invention actually shortens the overall process time, the somewhat higher temperatures can be used by a careful balancing of all of the process parameters.

A preferred example of the present invention operation utilizes the flushing action by having the steam injection into the bottom of the vessel, the application of vacuum from the top of the vessel, and the knife action at 1,750 r.p.m. all occurring simultaneously. When steam is visible in the exhaust line, typically after about 40 seconds, the vacuum is shut off, while the steam injection is continued for typically another 6 minutes, plus or minus 30 seconds, to achieve the required sterility of vessel content at 272° F. before it is shut off. Throughout this heat-up time, the knives keep running at 1,750 r.p.m.

There are several advantages to these modifications. Operating with a knife speed of 3,500 r.p.m. is eliminated. This modification increases the consistency of the sterilizing process and shortens the process cycle time by 3 to 5 minutes, amounting to an increase in performance of 4% to 5%. In terms of energy costs and safety of operation, that is a significant and important advance over the original process.

1. In the process for reducing medical waste to a sterile ash in an autoclave having disintegration equipment contained therein in which waste is disintegrated and heated in said autoclave and thereafter exhausting the autoclave contents, the improvement which comprises injecting steam into the bottom of said autoclave, drawing a vacuum from the to of the autoclave, and conducting said disintegration simultaneously until steam is visible from the vacuum exhaust line, at which time the vacuum is shut off, but the steam injection and disintegration continues until the desired level of sterility is obtained.
